SEQUENCE LISTING (2 sequences)

SEQ ID NO: 1 - protein, 10 aa, Homo sapiens
  MISC_FEATURE (1)..(1): Xaa is either Ala or Ile
XQTTXXXXXE

SEQ ID NO: 2 - protein, 10 aa, Homo sapiens
  MISC_FEATURE (2)..(2): Xaa is Gln or Met
AXTTXXAXXE

(19) United States
(12) Patent Application Publication
(10) Pub. No.: US 2012/0165272 A1
(43) Pub. Date: Jun. 28, 2012

(54) Tear Substitutes

(75) Inventors: Jan Holgersson, Vastra Frolunda (SE); Anki Nilsson, Johanneshov (SE); Nathalie Chatzissavidou, Huddinge (SE); Tomas Johansson, Huddinge (SE)

(73) Assignee: Recopharma AB, Huddinge (SE)

(21) Appl. No.: 13/332,841

(22) Filed: Dec. 21, 2011

Related U.S. Application Data
(60) Provisional application No. 61/425,524, filed on Dec. 21, 2010.

(51) Int. Cl. A61K 38/17; A61P 27/02; C07K 19/00

(52) U.S. Cl. 514/20.8; 530/391.7

(57) ABSTRACT

The invention features ophthalmic formulations of mucin polypeptides to treat or prevent dry eye.

### RELATED APPLICATIONS

This patent application claims priority from U.S. Provisional Application No. 61/425,524, filed on Dec. 21, 2010, the content of which is incorporated herein by reference in its entirety.

### FIELD OF THE INVENTION

This invention relates generally compositions containing recombinant mucins useful as tear substitutes for the treatment of dry eye.

### BACKGROUND OF THE INVENTION

The tear film is an aqueous solution containing electrolytes and proteins (the four major ones being: lysozyme, lactoferrin, lipocalin and secretory IgA), and with a lipid layer derived from the meibomian glands at the water-gas interphase (1). A very important protein constituent of the tear fluid is a group of proteins known as mucins. They are known to be secreted (MUC2, SAC, 5B, 6, 7, 9) or membrane-associated (MUC1, 3A, 3B, 4, 16). Of the former, MUCSAC has been identified in the tear fluid (2) and the mRNA for MUCSAC, 5B, 6 and 7 but not MUC2 was detected in lacrimal gland tissue (3). The membrane-associated mucins MUC1, 3A, 3B, 4, 16 have been reported to be expressed by corneal and conjunctival epithelia, the lacrimal apparatus and in the tear fluid (2). The mucins contribute to the tear fluid's ability to protect the corneal and conjunctival cells from desiccation and abrasive stress. Further, one of the secreted mucins, the MUCSAC, is a large, gel-forming mucin that can trap foreign bodies and contribute to their clearance from the eye via the nasolacrimal duct. The membrane-bound mucins are important components of the glycocalyx; a protective layer anchored to the actin cytoskeleton of the corneal and conjunctival cells and which reaches roughly 200 nm out from the cell surface. In addition to its protective role, it is believed to interact with and anchor the mucins of the aqueous phase of the tear film. Abundant O-glycan structures on the mucins are believed to act as decoy receptors for viruses and bacteria attaching to host cells via carbohydrate-specific receptors (adhesins). Thus, the difference in the repertoire of carbohydrate structures on the cell surface and the soluble mucins in the tear film, will determine the eye's susceptibility to a particular pathogen.

The dry eye is a condition explained by an insufficient quantity, quality or stability of the tear film (4). Dry eye conditions can be classified into tear deficient or evaporative (4). The former is further subdivided into into the Sjogren syndrome-related (primary and secondary) and the non-Sjogren tear deficient (lacrimal disease, lacrimal obstruction and malfunctioning blinking reflex) conditions (4). The evaporative dry eye conditions are subdivided into oil deficient, lid related and those caused by a change to the ocular surface (4). Most commonly the aqueous deficient dry eye is associated with reduced tear production and the evaporative dry eye is usually caused by a meibomian gland malfunction (4). In an earlier attempt to categorize the dry eye disease the condition was divided into five groups: mucin deficiency, lipid deficiency, aqueous deficiency, eyelid abnormalities or inadequate blink function, and ocular surface abnormality (5).

Because the tear film contributes to the lubrication and hydration of contact lenses, a normal tear film is required for problem-free contact lens wear. Further, contact lens wear can precipitate a subclinical dry eye condition. Thus, tear fluid substitutes may be required for successful lens wear.

Artificial tears may be classified according to chemical composition or biological effects. The following constituents have been used in artificial tears (for a comprehensive list of constituents in various commercially available tear substitutes see (6)): 1) water, 2) saline solutions, 3) glycerol, monosaccharides and disaccharides (e.g. glycerol, sucrose, dextrose, sorbitol, mannitol), 4) polysaccharides (e.g. mucilages [gums], dextrans and mucopolysaccharides [sodium hyaluronate, sodium chondroitin sulfate]), 5) synthetic polymers (e.g. vinyl derivatives, ethylene glycol derivatives, other synthetic polymers [polysorbates]), 6) gelatins, 7) biological fluids (e.g. serum, colostrum, saliva, egg whites, and mucins), and 8) lipids (6). None of the biological fluids are commercialized (6).

### SUMMARY OF THE INVENTION

The invention provides an ophthalmic formulation comprising an amount of a recombinant mucin polypeptide. The recombinant mucin is present in the formulation in an effective to treat or prevent dry eye. Optionally, the formulation contains a pharmaceutically acceptable carrier.

The pharmaceutically acceptable carrier contains one or more ingredients selected from the group consisting of surfactants; tonicity agents; buffers; preservatives; co-solvents; and viscosity building agents.

The recombinant mucin polypeptide is for example, PSGL-1, CD34, CD43, CD45, CD96, GlyCAM-1, and MAdCAM-1 or fragment thereof. For example the mucin polypeptide comprises at least a region of PSGL-1, such as the extracellular portion. Alternatively the recombinant mucin polypeptide is a secreted mucin or a membrane associated mucin. The secreted mucin is MUC2, MUC5AC, MUC5B, MUC6, MUC7 or MUC9. The membrane associated mucin is MUC1, MUC3A, MUC3B, MUC4, or MUC16.

In some aspects the recombinant mucin is glycosylated by one or more glycosyltransferases. For example, the recombinant mucin is sialylated. In some embodiments the multiple recombinant mucins are cross-linked such that the molecular weight is greater than 1000 kDa. In some aspects the recombinant mucin polypeptide is covalently linked to at least a region of an immunoglobulin polypeptide, such as a region of a heavy chain immunoglobulin polypeptide. Preferably the immunoglobulin polypeptide is an Fc region of an immunoglobulin heavy chain.

Also included in the invention are methods of treating a subject having dry eye, by administering to the eye surface of the subject an ophthalmic formulation the invention

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION

The present invention is directed to ophthalmic preparations for use as a tear film supplement. More specifically, this application relates to an aqueous formulation to be instilled into the eye, or in which to pre soak or store an object to be inserted into the eye, such as a contact lens, an ointment, or a solid device to be inserted into the conjunctival sac.

In particular the present invention relates to an ophthalmic pharmaceutical composition for treating and/or preventing the ophthalmologic clinical symptoms and signs in keratoconjunctivitis sicca or dry eye syndrome, which comprises a recombinant mucin polypeptide as an effective ingredient.

The preparations are useful for the treatment of disorders such as keratoconjunctivitis sicca or dry eye syndrome. In general, the preparations are also effective for the relief of symptoms of eye irritation, such as those caused by dry environmental conditions or by contact lens wear.

Definitions

The term “acute” as used herein denotes a condition having a rapid onset, and symptoms that are severe but short in duration.

[The term “analgesic” as used herein denotes a compound/formulation for the management of intermittent and/or chronic physical discomfort, suitable for long term use.

The term “anesthetic” or “anesthesia” as used herein denotes a compound/formulation for the management of acute physical pain, suitable for short term, temporary use, which has an effect that produces numbing or decreased sensitivity in the body part/organ to which the compound/formulation is administered (e.g., decreased corneal sensitivity of the eye).

The term “aqueous” typically denotes an aqueous composition wherein the carrier is to an extent of >50%, more preferably >75% and in particular 90% by weight water. [The term “chronic” as defined herein is meant a persistent, lasting condition, or one marked by frequent recurrence, preferably a condition that persists/recurs for greater than 3 months, more preferably greater than 6 months, more preferably greater than 12 months, and even more preferably greater than 24 months.

The term “comfortable” as used herein refers to a sensation of physical well being or relief, in contrast to the physical sensation of pain, burning, stinging, itching, irritation, or other symptoms associated with physical discomfort.

The term “comfortable ophthalmic formulation” as used herein refers to an ophthalmic formulation which provides physical relief from symptoms associated with dry eye disease and/or ocular discomfort, and only causes an acceptable level of pain, burning, stinging, itching, irritation, or other symptoms associated with ocular discomfort, when instilled in the eye, which are less than those seen with dosing with current concentrations on the market.

The term “dry eye” as used herein, refers to inadequate tear production and/or abnormal tear composition. Causes of dry eye disease as defined herein include but are not limited to the following: idiopathic, congenital alacrima, xerophthalmia, lacrimal gland ablation, and sensory denervation; collagen vascular diseases, including rheumatoid arthritis, Wegener's granulomatosis, and systemic lupus erythematosus; Sjogren's syndrome and autoimmune diseases associated with Sjogren's syndrome; abnormalities of the lipid tear layer caused by blepharitis or rosacea; abnormalities of the mucin tear layer caused by vitamin A deficiency; trachoma, diphtheric keratoconjunctivitis; mucocutaneous disorders; aging; menopause; and diabetes. Dry eye signs and/or symptoms as defined herein may also be provoked by other circumstances, including but not limited to the following: prolonged visual tasking; working on a computer; being in a dry environment; ocular irritation; contact lenses, LASIK and other refractive surgeries; fatigue; and medications such as isotretinoin, sedatives, diuretics, tricyclic antidepressants, antihypertensives, oral contraceptives, antihistamines, nasal decongestants, beta-blockers, phenothiazines, atropine, and pain relieving opiates such as morphine.

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a sign and/or symptom of dry eye and/or eye irritation, or prevent or treat dry eye and/or eye irritation. The effective amount may vary depending on such factors as the disease or condition being treated, the particular composition being administered, or the severity of the disease or condition. One of skill in the art may empirically determine the effective amount of a particular agent without necessitating undue experimentation.

A “patient,” “subject,” or “host” to be treated by the subject method refers to either a human or non-human animal, such as a primate, mammal, and vertebrate

The phrase “pharmaceutically acceptable” is art-recognized and refers to compositions, polymers and other materials and/or salts thereof and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the surface of the eye. Each carrier must be “acceptable” in the sense of being compatible with the other ingredients of the composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, hydroxypropylmethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) gums such as HP-guar; (22) polymers; and (23) other non-toxic compatible substances employed in pharmaceutical formulations.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, ptoluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; tromethamine, and the like. See, e.g., J. Pharm. Sci., 66: 1-19 (1977).

The term “preventing,” when used in relation to a condition, such as dry eye and/or eye irritation, is art-recognized, and refers to administration of a composition which reduces the frequency of, or delays the onset of, signs and/or symptoms of a medical condition in a subject relative to a subject which does not receive the composition.

As used herein, the terms “tear substitute” and “artificial tear” may be used interchangeably, and each refers to one or more molecules or compositions, which lubricate, “wet,” approximate the consistency of endogenous tears, aid in natural tear build up, or otherwise provide temporary relief of dry eye signs and/or symptoms and conditions upon ocular administration, including without limitation a polymer (e.g., a cellulosic polymer), an ocular surface protectant, a demulcent, or other component found on the FDA monograph for tear substitutes. The term “tear substitute component” refers to one or more components thereof.

The term “treating” is an art-recognized term which refers to reducing or ameliorating at least one sign and/or symptom of any condition or disease.

Mucin Polypeptides

In various aspects the invention provides composition containing a recombinant mucin polypeptide useful for the treatment of dry eye.

A “mucin polypeptide” refers to a polypeptide having a mucin domain. The mucin polypeptide has one, two, three, five, ten, twenty or more mucin domains. The mucin polypeptide is any glycoprotein characterized by an amino acid sequence substituted with O-glycans. For example, a mucin polypeptide has every second or third amino acid being a serine or threonine. The mucin polypeptide is a secreted protein. Alternatively, the mucin polypeptide is a cell surface protein.

Mucin domains are rich in the amino acids threonine, serine and proline, where the oligosaccharides are linked via N-acetylgalactosamine to the hydroxy amino acids (O-glycans). A mucin domain comprises or alternatively consists of an O-linked glycosylation site. A mucin domain has 1, 2, 3, 5, 10, 20, 50, 100 or more O-linked glycosylation sites. Alternatively, the mucin domain comprises an N-linked glycosylation site. A mucin polypeptide has 50%, 60%, 80%, 90%, 95% or 100% of its mass due to the glycan. A mucin polypeptide is any polypeptide encode for by a MUC genes (i.e., MUC1, MUC2, MUC3, MUC4, MUC5a, MUC5b, MUC5c, MUC6, MUC11, MUC12, etc.). Alternatively, a mucin polypeptide is P-selectin glycoprotein ligand 1 (PSGL-1), CD34, CD43, CD45, CD96, GlyCAM-1, MAdCAM-1, red blood cell glycophorins, glycocalicin, glycophorin, sialophorin, leukosialin, LDL-R, ZP3, and epiglycanin. Preferably, the mucin is PSGL-1. PSGL-1 is a homodimeric glycoprotein with two disulfide-bonded 120 kDa subunits of type 1 transmembrane topology, each containing 402 amino acids. In the extracellular domain there are 15 repeats of a 10-amino acid consensus sequence that contains 3 or 4 potential sites for addition of O-linked oligosaccharides. In one embodiment, the 10-amino acid consensus sequence is A(I) Q T T Q(PAR) P(LT) A(TEV) A(PG) T(ML) E (SEQ ID NO: 1). In another embodiment, the 10-amino acid consensus sequence is A Q(M) T T P(Q) P(LT) A A(PG) T(M) E (SEQ ID NO: 2). PSGL-1 is predicted to have more than 53 sites for O-linked glycosylation and 3 sites for N-linked glycosylation in each monomer.

The mucin polypeptide contains all or a portion of the mucin protein. Alternatively, the mucin protein includes the extracellular portion of the polypeptide. For example, the mucin polypeptide includes the extracellular portion of PSGL-1 or a portion thereof (e.g., amino acids 19-319 disclosed in GenBank Accession No. A57468). The mucin polypeptide also includes the signal sequence portion of PSGL-1 (e.g., amino acids 1-18), the transmembrane domain (e.g., amino acids 320-343), and the cytoplamic domain (e.g., amino acids 344-412).

The recombinant mucin polypeptides may exist as oligomers, such as dimers, trimers or pentamers. Preferably, the fusion polypeptide is a dimer.

A “non-mucin polypeptide” refers to a polypeptide of which at least less than 40% of its mass is due to glycans.

The mucin polypeptide corresponds to all or a portion of a mucin protein. For example, the recombinant mucin polypeptide comprises at least a portion of a mucin protein. “At least a portion” is meant that the mucin polypeptide contains at least one mucin domain (e.g., an O-linked glycosylation site). The mucin protein comprises the extracellular portion of the polypeptide. For example, the mucin polypeptide comprises the extracellular portion of PSGL-1.

The recombinant mucin polypeptide is glycosylated by one or more glycosyltransferases. The first polypeptide is glycosylated by 2, 3, 5 or more glycosyltransferases. Glycosylation is sequential or consecutive. Alternatively glycosylation is concurrent or random, i.e., in no particular order. The first polypeptide is glycosylated by any enzyme capable of adding or producing N-linked or O-linked glycans to or on a protein backbone. For example the first polypeptide is glycosylated by α2,3- and/or α2,6-sialyltransferase. Suitable sources for α2,3/6-sialyltransferase include but are not limited to GenBank Accession Nos. NP_{—}059132, AA039150, ABP35533, ABP35532, ABQ10741, ABQ10740, AAS77221, AAS77220, AAS77219, AAS77216, AAS77215, AAS77214, AAX20109, AA039151, AA039149, AAP47170, AAP47169, AAP47168, AAP47167, AAP47166, AAP47165, and AAP47164, and are incorporated herein by reference in their entirety. In a particular embodiment, the first polypeptide is glycosylated by both α2,3/6-sialyltransferase and core 2 β1,6-N-acetylglucosaminyltransferase. Suitable sources for core 2 β1,6-N-acetylglucosaminyltransferase include but are not limited to GenBank Accession Nos. CAA79610, Z19550, BAB66024, AP001515, AJ420416.1, AK313343.1, AL832647.2, AY196293.1, BC074885.2, BC074886, BC109101, BC109102.1, M97347.1, BAG36146.1, CAD89956.1, AAH74885.1, AAH74886.1, AAI09102.1, AAI09103.1, AAA35919.1, AAH17032, 095395, NP_{—}004742, EAW77572, NP_{—}004742.1, BC017032, AF102542.1, AAD10824.1, AF038650.1, NM_{—}004751.2, Q9P109, NP_{—}057675, EAW95751, AF132035.1, AAF63156.1, and NP_{—}057675.1. The first polypeptide contains greater than 40%, 50%, 60%, 70%, 80%, 90% or 95% of its mass due to carbohydrate.

In some aspect the recombinant mucin polypeptide is operatively linked to a second polypeptide. As used herein, a “fusion protein” or “chimeric protein” includes at least a portion of a mucin polypeptide operatively linked to a non-mucin polypeptide.

Within the fusion protein, the term “operatively linked” is intended to indicate that the mucin polypeptide and second polypeptides are chemically linked (most typically via a covalent bond such as a peptide bond) in a manner that allows for O-linked and/or N-linked glycosylation of the mucin polypeptide. When used to refer to nucleic acids encoding a fusion polypeptide, the term operatively linked means that a nucleic acid encoding the mucin polypeptide and the non-mucin polypeptide are fused in-frame to each other. The non-mucin polypeptide can be fused to the N-terminus or C-terminus of the mucin polypeptide.

Optionally, the mucin fusion polypeptide is linked to one or more additional moieties. For example, the fusion protein may additionally be linked to a GST fusion protein in which the fusion protein sequences are fused to the C-terminus of the GST (i.e., glutathione S-transferase) sequences. Such fusion proteins can facilitate the purification of the fusion protein. Alternatively, the fusion protein may additionally be linked to a solid support. Various solid supports are known to those skilled in the art. For example, the fusion protein is linked to a particle made of, e.g., metal compounds, silica, latex, polymeric material; a microtiter plate; nitrocellulose, or nylon or a combination thereof.

The fusion protein includes a heterologous signal sequence (i.e., a polypeptide sequence that is not present in a polypeptide encoded by a mucin nucleic acid) at its N-terminus For example, the native mucin glycoprotein signal sequence can be removed and replaced with a signal sequence from another protein. In certain host cells (e.g., mammalian host cells), expression and/or secretion of polypeptide can be increased through use of a heterologous signal sequence.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene is synthesized by conventional techniques including automated DNA synthesizers. Alternatively, PCR amplification of gene fragments is carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that encode a fusion moiety (e.g., an Fc region of an immunoglobulin heavy chain). A mucin encoding nucleic acid can be cloned into such an expression vector such that the fusion moiety is linked in-frame to the immunoglobulin protein.

The fusion polypeptides may exist as oligomers, such as dimers, trimers or pentamers. Preferably, the fusion polypeptide is a dimer.

The mucin polypeptide, and/or nucleic acids encoding the mucin polypeptide, is constructed using mucin encoding sequences are known in the art. Suitable sources for mucin polypeptides and nucleic acids encoding mucin polypeptides include GenBank Accession Nos. NP663625 and NM145650, CAD10625 and AJ417815, XP140694 and XM140694, XP006867 and XM006867 and NP00331777 and NM009151 respectively, and are incorporated herein by reference in their entirety.

The mucin polypeptide moiety is provided as a variant mucin polypeptide having an alteration in the naturally-occurring mucin sequence (wild type) that results in increased carbohydrate content (relative to the non-mutated sequence). As used herein, an alteration in the naturally-occurring (wild type) mucin sequence includes one or more one or more substitutions, additions or deletions into the nucleotide and/or amino acid sequence such that one or more amino acid substitutions, additions or deletions are introduced into the encoded protein. Alterations can be introduced into the naturally-occurring mucin sequence by standard techniques, such as site-directed mutagenesis and PCR-mediated mutagenesis.

For example, the variant mucin polypeptide comprised additional O-linked glycosylation sites compared to the wild-type mucin. Alternatively, the variant mucin polypeptide comprises an amino acid sequence alteration that results in an increased number of serine, threonine or proline residues as compared to a wild type mucin polypeptide. This increased carbohydrate content can be assessed by determining the protein to carbohydrate ratio of the mucin by methods known to those skilled in the art.

Alternatively, the mucin polypeptide moiety is provided as a variant mucin polypeptide having alterations in the naturally-occurring mucin sequence (wild type) that results in a mucin sequence with more O-glycosylation sites or a mucin sequence preferably recognized by peptide N-acetylgalactosaminyltransferases resulting in a higher degree of glycosylation.

In some embodiments, the mucin polypeptide moiety is provided as a variant mucin polypeptide having alterations in the naturally-occurring mucin sequence (wild type) that results in a mucin sequence more resistant to proteolysis (relative to the non-mutated sequence).

The mucin polypeptide includes full-length PSGL-1. Alternatively, the first polypeptide comprise less than full-length PSGL-1 polypeptide, e.g., a functional fragment of a PSGL-1 polypeptide. For example the first polypeptide is less than 400 contiguous amino acids in length of a PSGL-1 polypeptide, e.g., less than or equal to 300, 250, 150, 100, or 50, contiguous amino acids in length of a PSGL-1 polypeptide, and at least 25 contiguous amino acids in length of a PSGL-1 polypeptide. The first polypeptide is, for example, the extracellular portion of PSGL-1, or includes a portion thereof. Exemplary PSGL-1 polypeptide and nucleic acid sequences include GenBank Access No: XP006867; XM006867; XP140694 and XM140694.

The second polypeptide is preferably soluble. In some embodiments, the second polypeptide includes a sequence that facilitates association of the fusion polypeptide with a second mucin polypeptide. The second polypeptide includes at least a region of an immunoglobulin polypeptide. “At least a region” is meant to include any portion of an immunoglobulin molecule, such as the light chain, heavy chain, Fc region, Fab region, Fv region or any fragment thereof. Immunoglobulin fusion polypeptide are known in the art and are described in e.g., U.S. Pat. Nos. 5,516,964; 5,225,538; 5,428,130; 5,514,582; 5,714,147; and 5,455,165.

The second polypeptide comprises a full-length immunoglobulin polypeptide. Alternatively, the second polypeptide comprises less than full-length immunoglobulin polypeptide, e.g., a heavy chain, light chain, Fab, Fab₂, Fv, or Fc. Preferably, the second polypeptide includes the heavy chain of an immunoglobulin polypeptide. More preferably the second polypeptide includes the Fc region of an immunoglobulin polypeptide.

The second polypeptide has less effector function than the effector function of an Fc region of a wild-type immunoglobulin heavy chain. Alternatively, the second polypeptide has similar or greater effector function of an Fc region of a wild-type immunoglobulin heavy chain. An Fc effector function includes for example, Fc receptor binding, complement fixation and T cell depleting activity (see for example, U.S. Pat. No. 6,136,310). Methods of assaying T cell depleting activity, Fc effector function, and antibody stability are known in the art. In one embodiment the second polypeptide has low or no affinity for the Fc receptor. Alternatively, the second polypeptide has low or no affinity for complement protein Clq.

Another aspect of the invention pertains to vectors, preferably expression vectors, containing a nucleic acid encoding mucin polypeptides, or derivatives, fragments, analogs or homologs thereof. The vector contains a nucleic acid encoding a mucin polypeptide operably linked to a nucleic acid encoding an immunoglobulin polypeptide, or derivatives, fragments analogs or homologs thereof. Additionally, the vector comprises a nucleic acid encoding a glycosyltransferase such as an α2,3- and/or α2,6-sialyltransferase. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of fusion polypeptides in prokaryotic or eukaryotic cells. For example, fusion polypeptides can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRITS (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

The fusion polypeptide expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, fusion polypeptide can be expressed in insect cells using baculovirus expression vectors. Baculovirus vectors available for expression of proteins in cultured insect cells (e.g., Mamestra brassicae cells or SF9 cells) include the pAc series (Smith, et al., 1983. Mol. Cell. Biol. 3: 2156-2165) and the pVL series (Lucklow and Summers, 1989. Virology 170: 31-39).

A nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, fusion polypeptides can be expressed in bacterial cells such as E. coli, insect cells such as M. brassicae, yeast or mammalian cells (such as human, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the fusion polypeptides or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) fusion polypeptides. Accordingly, the invention further provides methods for producing fusion polypeptides using the host cells of the invention. In one embodiment, the method comprises culturing the host cell of invention (into which a recombinant expression vector encoding fusion polypeptides has been introduced) in a suitable medium such that fusion polypeptides is produced. In another embodiment, the method further comprises isolating polypeptide from the medium or the host cell.

The fusion polypeptides may be isolated and purified in accordance with conventional conditions, such as extraction, precipitation, chromatography, affinity chromatography, electrophoresis or the like. For example, the immunoglobulin fusion proteins may be purified by passing a solution through a column which contains immobilized protein A or protein G which selectively binds the Fc portion of the fusion protein. See, for example, Reis, K. J., et al., J. Immunol. 132:3098-3102 (1984); PCT Application, Publication No. WO87/00329. The fusion polypeptide may then be eluted by treatment with a chaotropic salt or by elution with aqueous acetic acid (1 M).

Alternatively, the mucin polypeptide and or the fusion polypeptides according to the invention can be chemically synthesized using methods known in the art. Chemical synthesis of polypeptides is described in, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989). The polypeptides are purified so that they are substantially free of chemical precursors or other chemicals using standard peptide purification techniques. The language “substantially free of chemical precursors or other chemicals” includes preparations of peptide in which the peptide is separated from chemical precursors or other chemicals that are involved in the synthesis of the peptide. In one embodiment, the language “substantially free of chemical precursors or other chemicals” includes preparations of peptide having less than about 30% (by dry weight) of chemical precursors or non-peptide chemicals, more preferably less than about 20% chemical precursors or non-peptide chemicals, still more preferably less than about 10% chemical precursors or non-peptide chemicals, and most preferably less than about 5% chemical precursors or non-peptide chemicals.

Chemical synthesis of polypeptides facilitates the incorporation of modified or unnatural amino acids, including D-amino acids and other small organic molecules. Replacement of one or more L-amino acids in a peptide with the corresponding D-amino acid isoforms can be used to increase the resistance of peptides to enzymatic hydrolysis, and to enhance one or more properties of biologically active peptides, i.e., receptor binding, functional potency or duration of action. See, e.g., Doherty, et al., 1993. J. Med. Chem. 36: 2585-2594; Kirby, et al., 1993. J. Med. Chem. 36:3802-3808; Morita, et al., 1994. FEBS Lett. 353: 84-88; Wang, et al., 1993. Int. J. Pept. Protein Res. 42: 392-399; Fauchere and Thiunieau, 1992. Adv. Drug Res. 23: 127-159.

Introduction of covalent cross-links into a peptide sequence can conformationally and topographically constrain the polypeptide backbone. This strategy can be used to develop peptide analogs of the fusion polypeptides with increased potency, selectivity and stability. Because the conformational entropy of a cyclic peptide is lower than its linear counterpart, adoption of a specific conformation may occur with a smaller decrease in entropy for a cyclic analog than for an acyclic analog, thereby making the free energy for binding more favorable. Macrocyclization is often accomplished by forming an amide bond between the peptide N- and C-termini, between a side chain and the N- or C-terminus [e.g., with K₃Fe(CN)₆ at pH 8.5] (Samson et al., Endocrinology, 137: 5182-5185 (1996)), or between two amino acid side chains. See, e.g., DeGrado, Adv Protein Chem, 39: 51-124 (1988). Disulfide bridges are also introduced into linear sequences to reduce their flexibility. See, e.g., Rose, et al., Adv Protein Chem, 37: 1-109 (1985); Mosberg et al., Biochem Biophys Res Commun, 106: 505-512 (1982). Furthermore, the replacement of cysteine residues with penicillamine (Pen, 3-mercapto-(D) valine) has been used to increase the selectivity of some opioid-receptor interactions. Lipkowski and Carr, Peptides: Synthesis, Structures, and Applications, Gutte, ed., Academic Press pp. 287-320 (1995).

Ophthalmic Formulations

The invention features novel ophthalmic formulation comprising a recombinant mucin which is comfortable upon instillation to the ocular surface, and safe for repeated, chronic use. As such, the comfortable ophthalmic formulations described herein will treat signs and symptoms of dry eye and/or ocular irritation, and increase long term patient compliance in the use of such formulations for the treatment and/or prevention of signs and symptoms associated with dry eye disease and/or ocular discomfort.

The invention is also based, in part, on that a recombinant mucin alone may be effective to improve tear film stability (assessed as an increase in tear film break up time and the Ocular Protection Index) and improve overall ocular surface health (assessed as reduced corneal staining and conjunctival redness, increased corneal sensitivity, decreased blink rate, and improved visual performance).

As such, the formulations are comfortable upon instillation into the eye, and may be used for relief of acute or chronic dry eye disease, and are particularly suitable for both intermittent and long term use. The formulations of the invention can also be used to treat another eye disorder if it contains a drug for that disorder.

The amount of mucin in an ophthalmic formulation can vary greatly depending on the product type. For example, in contact lens related solutions the mucin concentration would vary from about 0.001% to 5.0% by weight. In dry eye preparations the mucin level could vary from about 0.1% to about 10.0% by weight. In a solid ocular insert delivery device the mucin level could range to about 90.0% or greater by weight. Within each type of preparation, the concentration might be varied, depending on such factors as the severity of the dry eye condition being treated, to enhance particular properties of the mucin solution. These ranges are for purpose of illustration and are not meant in any manner to limit the scope of the claims.

The exemplary ophthalmic compositions finds particular utility as lubricating eye drops, i.e., an artificial tear solution, a tear fluid supplement, a delivery vehicle for topical ophthalmic drug application. In most of these applications, the compositions are provided in a buffered, sterile aqueous solution. Typically, these solutions have a viscosity from about 1 to 100 cps. As a solution the compositions are dispensed in the eye in the form of an eye drop. It should be understood, however, that the compositions described herein may also be formulated as viscous liquids, i.e., viscosities from several hundred to several thousand cps, gels or ointments. In these applications the mucin component would be dispersed or dissolved in an appropriate vehicle such as Lubragel, GRR Lubricating Jelly or Karajel, all trademarked products of United-Guardian, Inc., Hauppauge, N.Y.

The exemplary compositions may also be formulated as solid ocular inserts that dissolve or erode over time when placed in the cul-de-sac of the eye.

Swelling-controlled release devices would consist of mucin homogeneously dispersed in a glassy polymer such as a water soluble cellulosic. When the insert is placed in the eye, the tear fluid begins to penetrate the matrix, followed by swelling, and finally dissolution, of the matrix. As this process occurs, mucin is released into the eye to provide relief of dry eye symptoms over a long period of time.

Erodible devices would again consist of mucin homogeneously dispersed in a polymer matrix. In this case, mucin is released by a chemical reaction (hydrolysis) that results in solubilization of the matrix polymer, usually at the surface of the device. Generally, the matrix material is a polyanhydride or a poly(ortho ester).

In another embodiment the mucin may be chemically modified or crosslinked to act as its own “matrix”, where mucin comprises the entire, or nearly entire, device, thus providing the maximum amount of mucin available to the eye.

Furthermore, in some contact lens related embodiments, the exemplary transmembrane or surface mucin disclosed herein may be incorporated into contact lens soaking and conditioning solutions as well as lubricating eye drops for contact lens wearers.

In another embodiment the mucin may be utilized in drug delivery. The most common and convenient method for delivery of ocular drugs is by way of topical eye drops. Generally, the solution vehicles employed are quickly diluted by the tear fluid and drain from the eye in a matter of minutes. This short residence time hinders the absorption and hence the bioavailability of the drug in the eye. Oftentimes the short residence time is overcome by greatly increasing the concentration of the drug to improve bioavailability. This often leads to significant undesirable side effects due to the systemic actions of many of the ocular drugs currently prescribed.

Much research has been done to improve the residence time of the drug vehicle at the ocular surface and also to promote interaction or association of the drug with the vehicle. One approach that has been commercialized is to utilize a crosslinked carboxy-functional polymer such as Carbopol®, supplied by B.F. Goodrich. The bioadhesive nature of this polymer has been the basis for controlled release ophthalmic formulations as described in U.S. Pat. No. 4,615,697 and U.S. Pat. No. 5, 188,826, both of which are incorporated by reference in their entirety.

These crosslinked carboxy-functional polymers swell in aqueous solution but remain as micron-size hydrated particles. Furthermore, at neutral pH, they are substantially anionic in nature. Since many ophthalmic drugs, for example timolol and pilocarpine, are positively charged, they will associate with the negatively charged polymer particles through electrostatic interaction. Also, since the hydrated particles are microporous, the drug can be absorbed into the matrix. When an ophthalmic solution of this type is placed in the eye, the hydrated polymer particles adhere to the mucosal surface, providing extended residency time. During this residence the drug is released from the hydrated polymer particles, thus providing for a more efficient local delivery to the eye.

The mucins, used in the exemplary compositions are by definition “bioadhesive” and contain multiple negative charges. Given this information one would expect the mucins of this invention to act in a similar manner to the crosslinked carboxy-functional polymers as an ophthalmic drug delivery vehicle. In practice, these transmembrane or surface mucins provide superior retention time due to their ability to interact not only with the epithelial surface but also with the natural mucins in the tear film.

Exemplary ophthalmic formulations includes recombinant mucins from any number of the exemplary sources described herein. In addition, other solution components may be employed as required:

Excipients

In some embodiments, the mucin formulations of the invention comprise one or more pharmaceutically acceptable excipients. The term excipient as used herein broadly refers to a biologically inactive substance used in combination with the active agents of the formulation. An excipient can be used, for example, as a solubilizing agent, a stabilizing agent, a surfactant, a demulcent, a viscosity agent, a diluent, an inert carrier, a preservative, a binder, a disintegrant, a coating agent, a flavoring agent, or a coloring agent. Preferably, at least one excipient is chosen to provide one or more beneficial physical properties to the formulation, such as increased stability and/or solubility of the active agent(s). A “pharmaceutically acceptable” excipient is one that has been approved by a state or federal regulatory agency for use in animals, and preferably for use in humans, or is listed in the U.S. Pharmacopia, the European Pharmacopia or another generally recognized pharmacopia for use in animals, and preferably for use in humans.

Further examples of excipients include certain inert proteins such as albumins; hydrophilic polymers such as polyvinylpyrrolidone; amino acids such as aspartic acid (which may alternatively be referred to as aspartate), glutamic acid (which may alternatively be referred to as glutamate), lysine, arginine, glycine, and histidine; fatty acids and phospholipids such as alkyl sulfonates and caprylate; surfactants such as sodium dodecyl sulphate and polysorbate; nonionic surfactants such as such as TWEEN®, PLURONICS®, or a polyethylene glycol (PEG) designated 200, 300, 400, or 600; a Carbowax designated 1000, 1500, 4000, 6000, and 10000; carbohydrates such as glucose, sucrose, mannose, maltose, trehalose, and dextrins, including cyclodextrins; polyols such as mannitol and sorbitol; chelating agents such as EDTA; and salt-forming counter-ions such as sodium.

Examples of carriers that may be used in the formulations of the present invention include water, mixtures of water and water-miscible solvents, such as C₁- to C₇-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100000 times the concentration of the active ingredient.

In a particular embodiment, the carrier is a polymeric, mucoadhesive vehicle. Examples of mucoadhesive vehicles suitable for use in the methods or formulations of the invention include but are not limited to aqueous polymeric suspensions comprising one or more polymeric suspending agents including without limitation dextrans, polyethylene glycol, polyvinylpyrolidone, polysaccharide gels, Gelrite®, cellulosic polymers, and carboxy-containing polymer systems. In a particular embodiment, the polymeric suspending agent comprises a crosslinked carboxy-containing polymer (e.g., polycarbophil). In another particular embodiment, the polymeric suspending agent comprises polyethylene glycol (PEG). Examples of cross-linked carboxy-containing polymer systems suitable for use in the stable ophthalmic mucin formulations of the invention include but are not limited to Noveon AA-1, Carbopol®, and/or DuraSite® (InSite Vision).

In particular embodiments, the mucin formulations of the invention comprise one or more excipients selected from among the following: a tear substitute, a tonicity enhancer, a preservative, a solubilizer, a viscosity enhancing agent, a demulcent, an emulsifier, a wetting agent, a sequestering agent, and a filler. The amount and type of excipient added is in accordance with the particular requirements of the formulation and is generally in the range of from about 0.0001% to 90% by weight.

Tear Substitutes

The term “tear substitute” refers to molecules or compositions which lubricate, “wet,” approximate the consistency of endogenous tears, aid in natural tear build-up, or otherwise provide temporary relief of dry eye signs or symptoms and conditions upon ocular administration. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxymethyl cellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. Many such tear substitutes are commercially available, which include, but are not limited to cellulose esters such as Bion Tears®, Celluvisc®, Genteal®, OccuCoat®, Refresh®, Systane®, Teargen II®, Tears Naturale®, Tears Natural II®, Tears Naturale Free®, and TheraTears®; and polyvinyl alcohols such as Akwa Tears®, HypoTears®, Moisture Eyes®, Murine Lubricating®, and Visine Tears®, Soothe®. Tear substitutes may also be comprised of paraffins, such as the commercially available Lacri-Lube@ ointments. Other commercially available ointments that are used as tear substitutes include Lubrifresh PM®, Moisture Eyes PM® and Refresh PM®.

In one preferred embodiment of the invention, the tear substitute comprises hydroxypropylmethyl cellulose (Hypromellose or HPMC). According to some embodiments, the concentration of HPMC ranges from about 0.1% to about 2% w/v, or any specific value within said range. According to some embodiments, the concentration of HPMC ranges from about 0.5% to about 1.5% w/v, or any specific value within said range. According to some embodiments, the concentration of HPMC ranges from about 0.1% to about 1% w/v, or any specific value within said range. According to some embodiments, the concentration of HPMC ranges from about 0.6% to about 1% w/v, or any specific value within said range. In a preferred embodiments, the concentration of HPMC ranges from about 0.1% to about 1.0% w/v, or any specific value within said range (i.e., 0.1-0.2%, 0.2-0.3%, 0.3-0.4%, 0.4-0.5%, 0.5-0.6%, 0.6-0.7%, 0.7-0.8%, 0.8-0.9%, 0.9-1.0%; about 0.2%, about 0.21%, about 0.22%, about 0.23%, about 0.24%, about 0.25%, about 0.26%, about 0.27%, about 0.28%, about 0.29%, about 0.30%, about 0.70%, about 0.71%, about 0.72%, about 0.73%, about 0.74%, about 0.75%, about 0.76%, about 0.77%, about 0.78%, about 0.79%, about 0.80%, about 0.81%, about 0.82%, about 0.83%, about 0.84%, about 0.85%, about 0.86%, about 0.87%, about 0.88%, about 0.89%, or about 0.90%).

For example, without limitation, a tear substitute which comprises hydroxypropyl methyl cellulose is GenTeal® lubricating eye drops. GenTeal® (CibaVision—Novartis) is a sterile lubricant eye drop containing hydroxypropylmethyl cellulose 3 mg/g and preserved with sodium perborate. Other examples of an HPMC-based tear are provided.

In another preferred embodiment, the tear substitute comprises carboxymethyl cellulose sodium. For example, without limitation, the tear substitute which comprises carboxymethyl cellulose sodium is Refresh® Tears. Refresh® Tears is a lubricating formulation similar to normal tears, containing a, mild non-sensitizing preservative, stabilised oxychloro complex (Purite®)), that ultimately changes into components of natural tears when used.

In a preferred embodiment, the tear substitute, or one or more components thereof, is an aqueous solution having a viscosity in a range which optimizes efficacy of supporting the tear film while minimizing blurring, lid caking, etc. Preferably, the viscosity of the tear substitute, or one or more components thereof, ranges from 1-150 centipoise (cpi), e.g., 5-150 cpi, 5-130 cpi, 30-130 cpi, 50-120 cpi, 60-115 cpi (or any specific value within said ranges). In a particular embodiment, the viscosity of the tear substitute, or one or more components thereof, is about 70-90 cpi, or any specific value within said range (for example without limitation, 85 cpi).

Viscosity may be measured at a temperature of 20° C.+/−1° C. using a Brookfield Cone and Plate Viscometer Model VDV-III Ultra⁺with a CP40 or equivalent Spindle with a shear rate of approximately 22.50+/−approximately 10 (1/sec), or a Brookfield Viscometer Model LVDV-E with a SC4-18 or equivalent Spindle with a shear rate of approximately 26+/−approximately 10 (1/sec). Alternatively, viscosity may be measured at 25° C.+/−1° C. using a Brookfield Cone and Plate Viscometer Model VDV-III Ultra⁺with a CP40 or equivalent Spindle with a shear rate of approximately 22.50+/−approximately 10 (1/sec), or a Brookfield Viscometer Model LVDV-E with a SC4-18 or equivalent Spindle with a shear rate of approximately 26+/−approximately 10 (1/sec).

In some embodiments, the tear substitute, or one or more components thereof is buffered to a pH 5.0 to 9.0, preferably pH 5.5 to 7.5, more preferably pH 6.0 to 7.0 (or any specific value within said ranges), with a suitable salt (e.g., phosphate salts). In some embodiments, the tear substitute further comprises one or more ingredients, including without limitation, glycerol, propyleneglycerol, glycine, sodium borate, magnesium chloride, and zinc chloride.

Salts, Buffers, and Preservatives

The formulations of the present invention may also contain pharmaceutically acceptable salts, buffering agents, or preservatives. Examples of such salts include those prepared from the following acids: hydrochloric, hydrobromic, sulfuric, nitric, phosphoric, maleic, acetic, salicylic, citric, boric, formic, malonic, succinic, and the like. Such salts can also be prepared as alkaline metal or alkaline earth salts, such as sodium, potassium or calcium salts. Examples of buffering agents include phosphate, citrate, acetate, and 2-(N-morpholino)ethanesulfonic acid (MES).

For the adjustment of the pH, preferably to a physiological pH, buffers may especially be useful. The pH of the present solutions should be maintained within the range of 4.0 to 8.0, more preferably about 5.5 to 7.5, more preferably about 6.0 to 7.0. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred. Generally, buffers will be used in amounts ranging from about 0.05 to 2.5 percent by weight, and preferably, from 0.1 to 1.5 percent.

In certain embodiments, the formulations additionally comprise a preservative. A preservative may typically be selected from a quaternary ammonium compound such as benzalkonium chloride, benzoxonium chloride or the like. Benzalkonium chloride is better described as: N-benzyl-N—(C₈-C₁₈alkyl)-N,N-dimethylammonium chloride. Further examples of preservatives include antioxidants such as vitamin A, vitamin E, vitamin C, retinyl palmitate, and selenium; the amino acids cysteine and methionine; citric acid and sodium citrate; and synthetic preservatives such as thimerosal, and alkyl parabens, including for example, methyl paraben and propyl paraben. Other preservatives include octadecyldimethylbenzyl ammonium chloride, hexamethonium chloride, benzethonium chloride, phenol, catechol, resorcinol, cyclohexanol, 3-pentanol, m-cresol, phenylmercuric nitrate, phenylmercuric acetate or phenylmercuric borate, sodium perborate, sodium chlorite, alcohols, such as chlorobutanol, butyl or benzyl alcohol or phenyl ethanol, guanidine derivatives, such as chlorohexidine or polyhexamethylene biguanide, sodium perborate, Polyquad®, Germal®II, sorbic acid and stabilized oxychloro complexes (e.g., Purite®). Preferred preservatives are quaternary ammonium compounds, in particular benzalkonium chloride or its derivative such as Polyquad (see U.S. Pat. No. 4,407,791), alkyl-mercury salts, parabens and stabilized oxychloro complexes (e.g., Purite®). Where appropriate, a sufficient amount of preservative is added to the ophthalmic composition to ensure protection against secondary contaminations during use caused by bacteria and fungi.

In particular embodiments, the mucin formulations of the invention comprise a preservative selected from among the following: benzalkonium chloride, 0.001% to 0.05%; benzethonium chloride, up to 0.02%; sorbic acid, 0.01% to 0.5%; polyhexamethylene biguanide, 0.1 ppm to 300 ppm; polyquatemium-1 (Omamer M)—0.1 ppm to 200 ppm; hypochlorite, perchlorite or chlorite compounds, 500 ppm or less, preferably between 10 and 200 ppm); stabilized hydrogen peroxide solutions, a hydrogen peroxide source resulting in a weight % hydrogen peroxide of 0.0001 to 0.1% along with a suitable stabilizer; alkyl esters of p-hydroxybenzoic acid and mixtures thereof, preferably methyl paraben and propyl paraben, at 0.01% to 0.5%; chlorhexidine, 0.005% to 0.01%; chlorobutanol, up to 0.5%; and stabilized oxychloro complex (Purite®) 0.001% to 0.5%.

In another embodiment, the topical formulations of this invention do not include a preservative. Such formulations would be useful for patients who wear contact lenses, or those who use several topical ophthalmic drops and/or those with an already compromised ocular surface (e.g. dry eye) wherein limiting exposure to a preservative may be more desirable.

Viscosity Enhancing Agents and Demulcents

In certain embodiments, viscosity enhancing agents may be added to the mucin formulations of the invention. Examples of such agents include polysaccharides, such as hyaluronic acid and its salts, chondroitin sulfate and its salts, dextrans, various polymers of the cellulose family, vinyl polymers, and acrylic acid polymers.

In certain embodiments, the mucin formulations of the invention comprise ophthalmic demulcents and/or viscosity enhancing polymers selected from one or more of the following: cellulose derivatives such as carboxymethycellulose (0.01 to 5%) hydroxyethylcellulose (0.01% to 5%), hydroxypropyl methylcellulose or hypromellose (0.01% to 5%), and methylcelluose (0.02% to 5%); dextran 40/70 (0.01% to 1%); gelatin (0.01% to 0.1%); polyols such as glycerin (0.01% to 5%), polyethylene glycol 300 (0.02% to 5%), polyethylene glycol 400 (0.02% to 5%), polysorbate 80 (0.02% to 3%), propylene glycol (0.02% to 3%), polyvinyl alcohol (0.02% to 5%), and povidone (0.02% to 3%); hyaluronic acid (0.01% to 2%); and chondroitin sulfate (0.01% to 2%).

Viscosity of the stable ophthalmic mucin formulations of the invention may be measured according to standard methods known in the art, such as use of a viscometer or rheometer. One of ordinary skill in the art will recognize that factors such as temperature and shear rate may effect viscosity measurement. In a particular embodiment, viscosity of the is measured at 20° C.+/−1° C. using a Brookfield Cone and Plate Viscometer Model VDV-III Ultra+ with a CP40 or equivalent Spindle with a shear rate of approximately 22.50+/−approximately 10 (1/sec), or a Brookfield Viscometer Model LVDV-E with a SC4-18 or equivalent Spindle with a shear rate of approximately 26+/−approximately 10 (1/sec). In another embodiment, viscosity of the ophthalmic formulations of the invention is measured at 25° C.+/−1° C. using a Brookfield Cone and Plate Viscometer Model VDV-III Ultra+ with a CP40 or equivalent Spindle with a shear rate of approximately 22.50+/−approximately 10 (1/sec), or a Brookfield Viscometer Model LVDV-E with a SC4-18 or equivalent Spindle with a shear rate of approximately 26+/−approximately 10 (1/sec).

Tonicity Enhancers

Tonicity is adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, Na1, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

Solubilizing Agents

The formulation may additionally require the presence of a solubilizer, in particular if one or more of the ingredients tends to form a suspension or an emulsion. Suitable solubilizers include, for example, tyloxapol, fatty acid glycerol polyethylene glycol esters, fatty acid polyethylene glycol esters, polyethylene glycols, glycerol ethers, polysorbate 20, polysorbate 80 or mixtures of those compounds. In a preferred embodiment, the solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. In another embodiment, the solubilizer is tyloxapol or a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient. Preferably, the solubilizer is not a cyclodextrin compound (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin).

Methods of Use

The invention features methods of treating and/or preventing the signs and symptoms associated with dry eye and/or eye irritation in a subject comprising use of the novel NSAID alone formulations or combined tear/NSAID formulations described above. For example, a method of treating and/or preventing dry eye and/or eye irritation may comprise administering to the eye surface of the subject in need thereof a formulation comprising a recombinant mucin.

Provided also are methods of increasing the tear film break-up time (TFBUT) of a subject's tear film, comprising administering to the eye surface of the subject in need thereof a formulation comprising a recombinant mucin, in a pharmaceutically acceptable carrier. Optionally, the ophthalmic formulation for increasing TFBUT may further comprise a tear substitute, or one or more components thereof.

Provided also are methods of increasing the ocular protection index (OPI) of a subject's eye, comprising administering to the eye surface of the subject in need thereof a formulation comprising a recombinant mucin, in a pharmaceutically acceptable carrier. Optionally, the ophthalmic formulation for increasing OPI may further comprise a tear substitute, or one or more components thereof.

Provided also are methods for improving, treating, relieving, inhibiting, preventing, or otherwise decreasing ocular discomfort in a subject comprising administering to the eye surface of the subject in need thereof a formulation comprising a recombinant mucin in a pharmaceutically acceptable carrier. Optionally, the ophthalmic formulation for improving, treating, relieving, inhibiting, preventing, or otherwise decreasing ocular discomfort may further comprise a tear substitute, or one or more components thereof.

Provided also are method of improving overall ocular surface health of a subject's eye, comprising administering to the eye surface of the subject in need thereof a formulation comprising a low dose amount of at least one recombinant mucin in a pharmaceutically acceptable carrier. Optionally, the ophthalmic formulation for increasing OPI may further comprise a tear substitute, or one or more components thereof.

The effective amount of the one or more recombinant mucins in the ophthalmic formulations of the invention will depend on absorption, inactivation, and excretion rates of the drug as well as the delivery rate of the compound from the formulation, and will be suitable for short or long term use for the treatment of acute or chronic conditions, respectively. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of the recombinant mucin of the present invention will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment. The effectiveness of any formulation and method of treatment or prevention may be assessed by administering the formulation and assessing the effect of the administration by measuring one or more indices associated with the efficacy of the composition and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determine the optimum time and/or amount of administration, which will require no more than routine experimentation consisting of monitoring the subject and adjusting the dosage and/or timing

The combined use of several recombinant mucins formulated into the compositions of the present invention may reduce the required dosage for any individual component because the onset and duration of effect of the different components may be complimentary. In such combined therapy, the different recombinant mucins may be delivered together or separately, and simultaneously or at different times within the day.

Efficacy of the formulations and compositions of the invention in treating and preventing the signs and symptoms associated with dry eye disease and/or ocular irritation may be assessed by measuring changes in tear film break-up time (TFBUT), changes in ocular protection index (OPI), improved level of ocular comfort, decreased inflammation as measured by staining and/or redness, improved corneal sensitivity (e.g., as measured by Cochet-Bonnet test), decreased blink rate, improved visual acuity (e.g., as measured by the Inter-blink Interval Visual Acuity Decay (IVAD) test). An increase in TFBUT and/or OPI, and/or an improved level of ocular comfort, corneal sensitivity and/or visual acuity, and/or a decrease in the level of inflammation and/or blink rate in a subject following administration of the formulations and compositions of the invention to the ocular surface, as compared to the TFBUT, OPI, level of ocular discomfort, inflammation, corneal sensitivity, visual acuity, corneal staining and/or blink rate prior to administration to the ocular surface, indicates that the formulation is effective in treating and preventing signs and symptoms associated with dry eye disease and/or ocular irritation.

The ophthalmic formulations of the present invention effectively enhance tear film stability. One measure of tear film stability is an increase in tear film break up time (TFBUT) when measured post-instillation of the ophthalmic formulation into the eye as compared to TFBUT measured prior to instillation of the ophthalmic formulation into the eye (i.e., baseline TFBUT). For example, without limitation, TFBUT is increased by approximately 0.5 to 10 seconds or more (or any specific value within said range) post-instillation as compared to baseline TFBUT. More particularly, TFBUT is increased by about 0.5 seconds, about 1 second, about 1.5 seconds, about 2 seconds, about 2.5 seconds, about 3 seconds, about 3.5 seconds, about 4 seconds, about 4.5 seconds, about 5 seconds, about 5.5 seconds, about 6 seconds, about 6.5 seconds, about 7 seconds, about 7.5 seconds, about 8 seconds, about 8.5 seconds, about 9 seconds, about 9.5 seconds, about 10 seconds, or more, when measured post instillation as compared to baseline TFBUT.

One method of determining a clinically meaningful increase in TFBUT is an increase (i.e., improvement) in Ocular Protection Index (OPI) when measured post-instillation of the ophthalmic formulation into the eye as compared to OPI measured prior to instillation of the ophthalmic formulation into the eye (i.e., baseline OPI). This approach to measuring clinically relevant alterations in TFBUT, known as the Ocular Protection Index (OPI) has proven useful in assessing factors that cause dry eye and evaluating its therapeutic agents.

When studying the relationship between TFBUT and the inter-blink interval (IBI=time between complete blinks), it may be suggested that their interaction assists in regulating the integrity of an ocular surface. A protected surface exists when the TFBUT is longer than the IBI. In contrast, an unprotected surface exists when the TFBUT is shorter than the IBI. Studies have shown that within one second of TFBUT, patients report ocular discomfort and shortly thereafter develop superficial punctate keratitis. To prevent these symptoms and signs, the TFBUT must match or exceed the inter-blink period, providing complete protection of the ocular surface. When quantifying an agent's effect on tear film stability, a binomial analysis may be performed. The index allows for two possible outcomes after treatment, 1) success=TFBUT either matches or exceeds the inter-blink period so that the ocular surface is protected and 2) failure=TFBUT remains shorter than the inter-blink period so that the ocular surface is unprotected. An OPI score 1 is considered favorable since the patient has a tear protected ocular surface, resulting in fewer signs and symptoms associated with dry eye. An OPI score <1 is considered unfavorable since the patient has an exposed ocular surface, resulting in more signs and symptoms associated with dry eye.

The ophthalmic formulations of the invention effectively increase (i.e., improve) OPI. For example, without limitation, OPI is improved by about 0.1 to 10, or more (or any specific value within said range) when measured post-instillation of the ophthalmic formulation into the eye as compared to baseline OPI. More particularly, OPI is improved whereby the OPI is increased by about 0.1, 0.2, 0.3, 0.4, 0.5, 0.6, 0.7, 0.8, 0.9, 1.0 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, or more, when measured post instillation as compared to baseline OPI. Ocular irritation/discomfort is effectively decreased whereby patient assessment of ocular discomfort is less when measure post-instillation of the ophthalmic formulation into the eye as compared to ocular discomfort measured prior to instillation of the ophthalmic formulation into the eye.

TFBUT may be measured using various methods, including but not limited to illumination of the eye following instillation of sodium fluorescein in the eye, or equivalents thereof. OPI may be obtained by dividing the TFBUT by the time in seconds between blinks (the inter-blink interval, or “IBI”)

An increase in ocular comfort or decrease in ocular discomfort in a subject following administration of the formulations and compositions of the invention as compared to ocular comfort level prior to administration, indicates that the formulation is effective in treating and preventing signs and symptoms associated with dry eye limited to subjective scales (for example but not limited to, standardized subjective scales that determine ocular discomfort as mild, moderate, sever, or 0, 1, 2, 3, 4, etc., or other appropriate scale), reflexive response (e.g., flinch-reflex), and physiological response, including but not limited to changes in heart rate, blood pressure, and perspiration levels.

Efficacy of the formulations and compositions of the invention in improving overall ocular surface health may be assessed by measuring changes in corneal staining, conjunctival redness, corneal sensitivity, blink rate, and visual performance. Methods of assessing these parameters include: lissamine green or sodium fluorescein dyes, standardized assessment scales, Cochet Bonnet aesthesiometry or non-contact aesthesiometry, video recording and software analysis, and questionnaires or the Inter-blink Interval Visual Acuity Decay (IVAD) test, respectively.

Packaging

The formulations of the present invention may be packaged as either a single dose product or a multi-dose product. The single dose product is sterile prior to opening of the package and all of the composition in the package is intended to be consumed in one or several applications to one or both eyes of a patient. The use of an antimicrobial preservative to maintain the sterility of the composition after the package is opened is generally unnecessary. The formulations, if an ointment formulation, may be packaged as appropriate for an ointment, as is known to one of skill in the art.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and other publications by the Food and Drug Administration, and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an anti-microbial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation, or patients using multiple preserved products. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

While the formulations of this invention are preferably formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

Kits

In still another embodiment, this invention provides kits for the packaging and/or storage and/or use of the formulations described herein, as well as kits for the practice of the methods described herein. Thus, for example, kits may comprise one or more containers containing one or more ophthalmic solutions, ointments, gels, sustained release formulations or devices, suspensions or formulations, tablets, or capsules of this invention. The kits can be designed to facilitate one or more aspects of shipping, use, and storage.

The kits may optionally include instructional materials containing directions (i.e., protocols) disclosing means of use of the formulations provided therein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like. Such media may include addresses to internet sites that provide such instructional materials.

### EXAMPLES

Examples of ophthalmic formulations of the present invention, illustrating the composition and the method of making such solutions, are noted below.

### Example I

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al. (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out as in standard PCR Protocols: A Guide To Methods and Applications, Academic Press, San Diego, Calif. (1990). In situ PCR in combination with Flow Cytometry (FACS) can be used for detection of cells containing specific DNA and mRNA sequences (Testoni et al., Blood 1996, 87:3822.) Methods of performing RT-PCR are well known in the art.

Cell Culture

HeLa cells (American Type Culture Collection) are cultured as described in Czauderna, et al. (NAR, 2003. 31:670-82). Human keratinocytes are cultured at 37° C. in Dulbecco's modified Eagle medium (DMEM) containing 10% FCS. The mouse cell line, B16V (American Type Culture Collection), is cultured at 37° C. in Dulbecco's modified Eagle medium (DMEM) containing 10% FCS. Culture conditions are as described in (Methods Find Exp Clin Pharmacol. 1997, 19(4):231-9).

### Example II

Rabbit Model to Reverse the Onset of Dry Eye

Dry eye is created in rabbits by surgically closing the lacrimal gland excretory duct, and allowing the rabbits to remain untreated for at least four weeks. See Gilbard, J. P, 1996, “Dry Eye: phramcological approaches, effects, and progress” CLAO J. 22, 141-145. After confirming dry eye by Schirmer test, and ocular surface staining, formulation of the invention is instilled as a solution at concentrations of 0.01, 0.1, 1.0%, 5%, or 10% in neutral, isotonic buffered aqueous solution. The formulation is administered in one 50 microliter drop to the ocular surface up to 1-5 times a day, every day for 2-10 weeks. The symptoms of dry eye are monitored once a week for 2-10 weeks and an increase in Schirmer scores and/or a decrease in the amount of ocular surface staining indicates the efficacy of the formulation of the current invention in the treatment of dry eye disease.

### Example III

Vehicle Formulations and Exemplary Eye Drop Formulations

The aqueous eye drop formulation optionally contain various additives incorporated ordinarily, such as buffering agents (e.g., phosphate buffers, borate buffers, citrate buffers, tartarate buffers, acetate buffers, amino acids, sodium acetate, sodium citrate and the like), isotonicities (e.g., saccharides such as sorbitol, glucose and mannitol, polyhydric alcohols such as glycerin, concentrated glycerin, polyethylene glycol and propylene glycol, salts such as sodium chloride), preservatives or antiseptics (e.g., benzalkonium chloride, benzethonium chloride, p-oxybenzoates such as methyl p-oxybenzoate or ethyl p-oxybenzoate, benzyl alcohol, phenethyl alcohol, sorbic acid or its salts, thimerosal, chlorobutanol and the like), solubilizing aids or stabilizing agents (e.g., cyclodextrins and their derivative, water-soluble polymers such as polyvinyl pyrrolidone) surfactants such as polysorbate 80 (Tween 80)), pH modifiers (e.g., hydrochloric acid, acetic acid, phosphoric acid, sodium hydroxide, potassium hydroxide, ammonium hydroxide and the like), chelating agents (e.g., sodium edetate, sodium citrate, condensed sodium phosphate) and the like.

The eye drop formulation in the form of an aqueous suspension may also contain suspending agents (e.g., polyvinyl pyrrolidone, glycerin monostearate) and dispersing agents (e.g., surfactants such as tyloxapol and polysorbate 80, ionic polymers such as sodium alginate), in addition to the additives listed above, thereby ensuring that the eye drop formulation is a further uniform microparticulate and satisfactorily dispersed aqueous suspension.

The ophthalmic ointment may comprise a known ointment base, such as purified lanolin, petrolatum, plastibase, liquid paraffin, polyethylene glycol and the like.

### Example IV

The starting materials for preparation of the solution are as follows: Sodium chloride 6.55 g; Trisodium citrate monohydrate 7.35 g; Citric acid 0.035 g; EDTA Na₂ 0.050 g; Mannitol 1.800 g; Propylmethylcellulose 1.00 g; appropriate or required amount of mucin polypeptide.

The solution is prepared by adding sodium chloride, trisodium citrate, citric acid monohydrate, EDTA Na₂, mannitol and propylmethylcellulose in the amounts specified above to one liter of water. The foregoing constituents are dissolved and autoclaved at a pressure of 15 lbs. and a temperature of 120° C. and chilled to 4° C. Appropriate or required amount mucin polypeptide is dissolved in 500 mg of Tween™ 80 by gentle warming at about 50° C. and shaking by hand and transferred quantitatively to 1 liter of the above mixture under constant magnetic stirring. The mixture was stirred overnight in cold room (4° C.). A clear solution is obtained. This solution is stored in a refrigerator at about 4° C. till used. The final concentration of recombinant mucin polypeptide is determined by high pressure liquid chromatography on C-18 column, using a 95% methanol 5% H₂O mixture as the eluting solvent and monitoring the effluent spectrometrically at appropriate nanometers. The concentration of mucin polypeptide remains stable for at least six weeks. The solution is stored in a dark bottle at 4° C.

### Example V

The starting materials for preparation of the solution are as follows: Sucrose 76 g; Trisodium citrate monohydrate 7.35 g; Citric acid 0.035 g; EDTA Na₂ 0.050 g; Mannitol 1.8 g; required amount of mucin polypeptide; TWEEN™ 80 500 mg.

The mucin polypeptide-containing solution is prepared by adding sodium chloride, trisodium citrate, citric acid monohydrate, EDTA Na₂ and mannitol in the amounts specified above to one liter of water. The foregoing constituents are dissolved and autoclaved at a pressure of 15 lbs. and a temperature of 120° C. and chilled to 4° C. Appropriate or required amount of mucin polypeptide are dissolved in 500 mg of Tween™ 80 by gentle warming at about 50° C. and shaking by hand and transferred quantitatively to 1 liter of the above mixture under constant magnetic stirring. The mixture is stirred overnight in cold room. A clear solution is obtained. This solution is stored in a refrigerator at about 4° C. till used.

### Example VI

A 0.5-1.0 fluid oz. eye dropper bottle is filled with 15 ml of a sterile aqueous solution containing per 1 ml: Appropriate or required amount of mucin polypeptide; TWEEN™ 80; 0.5 mg Nacl; 6.85 mg Na₃ citrate monohydrate; 7.35 mg Citric acid; 0.031 mg EDTA Na₂; 0.05 mg Mannitol; 1.8 mg Q.S. water up to 1 ml.

Two drops of the solution are placed in one eye of an individual suffering from dry, irritated eyes 1-5 times a day, for a period of 2-10 weeks. An unpreserved normal saline solution is placed in the other eye using the same schedule for comparison purposes. The eye being treated shows a marked improvement in subjective comfort and appearance compared to the saline solution treated eye.

The use of the term “solution” in the aforementioned specification is not to be construed as meaning a true solution according to pure technical definition. It is rather to be construed as meaning a mixture which appears to the naked eye to be a solution, and accordingly, the word “solution” is to be construed as covering transparent emulsions of solubilized mucin polypeptide, its derivatives and precursors.

The foregoing detailed specification has been given for the purpose of explaining and illustrating the invention. It is to be understood that the invention is not limited to detailed information set forth, and that various modifications can be made. It is intended to cover such modifications and changes as would occur to one skilled in the art, as the following claims permit and consistent with the state of the prior art.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Tiffany J M. The normal tear film. In: Geerling G, Brewitt H, eds. Dev Ophtalmol, vol 41. Basel: Karger, 2008: 1.
2. Ramamoorthy P, Nichols J J. Mucins in contact lens wear and dry eye conditions. Optom Vis Sci 2008; 85: E631.
3. Paulsen F, Langer G, Hoffman W, Berry M. Human lacrimal gland mucins. Cell Tissue Res 2004; 316: 167.
4. Foulks G N. What is dry eye and what does it mean to the contact lens wearer? Eye & Contact Lens 2003; 29(1S): S96.
5. Holly F J, Lemp M A. Tear physiology and dry eyes. Sury Opthalmol 1977; 22: 69.
6. Murube J, Paterson A, Murube E. Classification of artificial tears. I: Composition and properties. Adv Exp Med Biol 1998; 438: 693.
7. Gustafsson A, Holgersson J. A new generation of carbohydrate-based therapeutics: recombinant mucin-type fusion proteins as versatile inhibitors of protein-carbohydrate interactions. Expert Opin. Drug Discov 2006; 1: 161.

What is claimed is:

1. An ophthalmic formulation comprising an amount of a recombinant mucin polypeptide effective to treat or prevent dry eye.

2. The ophthalmic formulation of claim 1, wherein the formulation further comprises a pharmaceutically acceptable carrier.

3. The ophthalmic formulation of claim 2, wherein the pharmaceutically acceptable carrier comprises one or more ingredients selected from the group consisting of surfactants; tonicity agents; buffers; preservatives; co-solvents; and viscosity building agents.

4. The ophthalmic formulation of claim 1, wherein the recombinant mucin polypeptide is PSGL-1, CD34, CD43, CD45, CD96, GlyCAM-1, MAdCAM-1, or fragment thereof.

5. The ophthalmic formulation of claim 4, wherein said mucin polypeptide comprises at least a region of a P-selectin glycoprotein ligand-1 (PSGL-1).

6. The ophthalmic formulation of claim of claim 5, wherein said mucin polypeptide includes an extracellular portion of a P-selectin glycoprotein ligand-1.

7. The ophthalmic formulation of claim 1, wherein the recombinant mucin polypeptide is a secreted mucin or a membrane associated mucin.

8. The ophthalmic formulation of claim 7, wherein the secreted mucin is MUC2, MUC5AC, MUC5B, MUC6, MUC7, or MUC9.

9. The ophthalmic formulation of claim 7, wherein the membrane associated mucin is MUC1, MUC3A, MUC3B, MUC4, or MUC16.

10. The ophthalmic formulation of claim 1, wherein said recombinant mucin is glycosylated by one or more glycosyltransferases.

11. The ophthalmic formulation of claim 1, wherein the recombinant mucin is sialylated.

12. The ophthalmic formulation of claim 1, wherein multiple recombinant mucins are cross-linked such that the molecular weight is greater than 1000 kDa.

13. The ophthalmic formulation of claim 1, wherein the recombinant mucin polypeptide is covalently linked to at least a region of an immunoglobulin polypeptide.

14. The ophthalmic formulation of claim 13, wherein the immunoglobulin polypeptide comprises a region of a heavy chain immunoglobulin polypeptide.

15. The ophthalmic formulation of claim 13, wherein the immunoglobulin polypeptide comprises an Fc region of an immunoglobulin heavy chain.

16. A method of treating a subject having dry eye, comprising administering to the eye surface of the subject an ophthalmic formulation of claim 1, wherein the formulation further comprises a pharmaceutically acceptable carrier comprising one or more ingredients selected from the group consisting of surfactants; tonicity agents; buffers; preservatives; co-solvents; and viscosity building agents; and wherein the recombinant mucin polypeptide is PSGL-1, CD34, CD43, CD45, CD96, GlyCAM-1, a secreted mucin, a membrane associated mucin, MAdCAM-1, or fragment thereof.
